(19) United States
(12) Patent Application Publication
(10) Pub. No.: US 2004/0107960 A1
(43) Pub. Date: Jun. 10, 2004

(54) Composite medical equipment having the phlegm extraction and mist spraying functions

(76) Inventors: Hung-Ming Wu, Taichung (TW); Fu-Chung Lai, Taichung (TW)

Correspondence Address: CHARLES E. BAXLEY, ESQUIRE, Hart, Baxley, Daniels & Holton, 59 John St, 5 Fl, New York, NY, 10038, US

(21) Appl. No.: 10/313,457

(22) Filed: Dec. 6, 2002

(51) Int. Cl. A61M 11/00

(52) U.S. Cl. 128/200.14

(57) ABSTRACT

A composite medical equipment having the phlegm extraction and mist spraying functions includes a compressor, a phlegm storage container, and a mist sprayer. Thus, the composite medical equipment can provide the phlegm extraction and mist spraying functions simultaneously, thereby reducing cost of the equipment, and thereby facilitating the user using the composite medical equipment.

### BACKGROUND OF THE INVENTION

1. Field of the Invention

The present invention relates to a composite medical equipment having the phlegm extraction and mist spraying functions, and more particularly to a composite medical equipment that can provide the phlegm extraction and mist spraying functions simultaneously, thereby reducing cost of the equipment, and thereby facilitating the user using the composite medical equipment.

2. Description of the Related Art

A conventional phlegm extractor is used to suck and draw the phlegm or dirt contained in the patient's throat, mouth, nose etc., thereby preventing the phlegm or dirt from choking the patient's respiratory duct. However, the conventional phlegm extractor produces a suction force, so that the patient's respiratory duct is dried during operation of the conventional phlegm extractor. Thus, the operator has to use a mist sprayer to wet the patient's respiratory duct during operation of the conventional phlegm extractor, thereby causing inconvenience in operation, and thereby increasing cost of the equipment.

### SUMMARY OF THE INVENTION

The present invention has arisen to mitigate and/or obviate the disadvantage of the conventional phlegm extractor.

The primary objective of the present invention is to provide a composite medical equipment having the phlegm extraction and mist spraying functions, wherein the composite medical equipment can provide the phlegm extraction and mist spraying functions simultaneously, thereby reducing cost of the equipment, and thereby facilitating the user using the composite medical equipment.

Another objective of the present invention is to provide a composite medical equipment having the phlegm extraction and mist spraying functions, wherein the pressure manometer mounted on the air extraction port of the compressor and the pressure manometer mounted on the air outlet of the compressor are used to measure the pressure of the gas flowing through the air extraction port and the air outlet of the compressor, thereby preventing the phlegm storage container and the mist sprayer from being inoperative due to the insufficient pressure force of the air extraction port and the air outlet of the compressor.

In accordance with the present invention, there is provided a composite medical equipment having the phlegm extraction and mist spraying functions, comprising a compressor, a phlegm storage container, and a mist sprayer, wherein:

the compressor includes a connection portion having a first end formed with an air extraction port and a second end formed with an air outlet;

the phlegm storage container is mounted on the compressor and is connected to the air extraction port of the compressor; and

the mist sprayer is mounted on the compressor and has a water chamber and a spraying nozzle, the mist sprayer is connected to the air outlet of the compressor.

Further benefits and advantages of the present invention will become apparent after a careful reading of the detailed description with appropriate reference to the accompanying drawings.

### BRIEF DESCRIPTION OF THE DRAWINGS

FIG. 1 is a front plan view of a composite medical equipment having the phlegm extraction and mist spraying functions in accordance with a preferred embodiment of the present invention;

FIG. 2 is a top plan view of the composite medical equipment having the phlegm extraction and mist spraying functions in accordance with the preferred embodiment of the present invention;

FIG. 3 is a schematic operational view of the composite medical equipment having the phlegm extraction and mist spraying functions as shown in FIG. 2 in use; and

FIG. 4 is a top plan view of the composite medical equipment having the phlegm extraction and mist spraying functions in accordance with another preferred embodiment of the present invention.

### DETAILED DESCRIPTION OF THE INVENTION

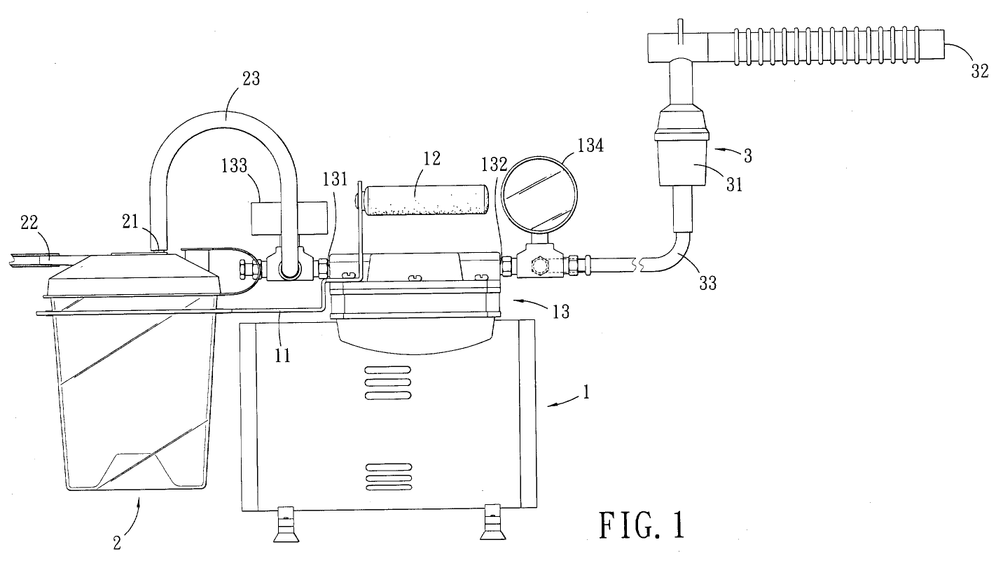

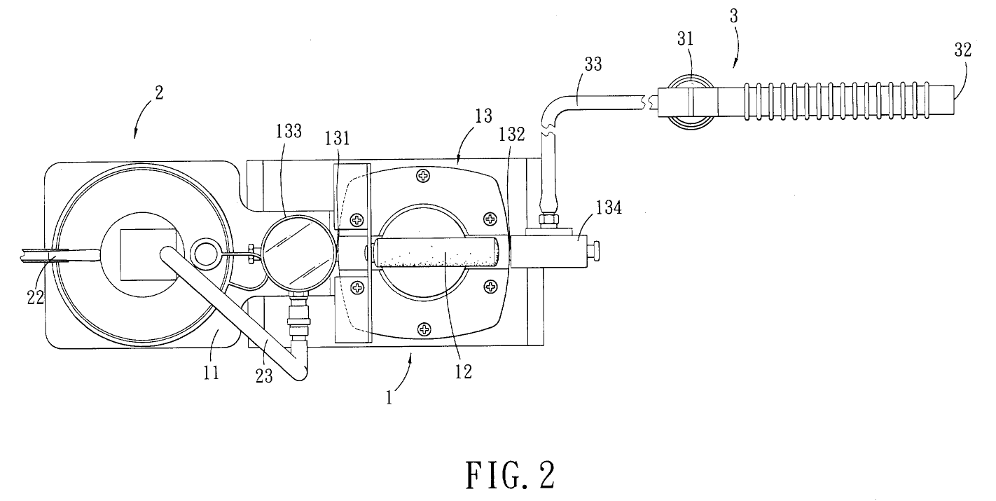

Referring to the drawings and initially to FIGS. 1 and 2, a composite medical equipment having the phlegm extraction and mist spraying functions in accordance with a preferred embodiment of the present invention comprises a compressor 1, a phlegm storage container 2, and a mist sprayer 3.

The compressor 1 can produce an air thrust force about two to three kilograms. The compressor 1 has a top provided with a support frame 11 and a handgrip 12. Thus, the user can hold the handgrip 12. The compressor 1 includes a connection portion 13 having a first end formed with an air extraction port 131 and a second end formed with an air outlet 132. The air outlet 132 of the compressor 1 can produce an air thrust force about two to three kilograms by the compressor 1. The compressor 1 also includes a pressure manometer 133 mounted on the air extraction port 131 and a pressure manometer 134 mounted on the air outlet 132.

The phlegm storage container 2 is mounted on the support frame 11 of the compressor 1 and has a top provided with a connection port 21 and a phlegm suction port 22. The connection port 21 of the phlegm storage container 2 is connected to the pressure manometer 133 of the air extraction port 131 of the compressor 1 by a tube 23. Thus, the connection port 21 of the phlegm storage container 2 is connected to the air extraction port 131 of the compressor 1. Thus, when the compressor 1 is operated, the phlegm storage container 2 produces a pressure about 550 to 600 millimeter mercury column (550 to 600 mmHg), so that the phlegm suction port 22 of the phlegm storage container 2 produces a suction force.

The mist sprayer 3 is mounted on the compressor 1, and has a water chamber 31 and a spraying nozzle 32. The mist sprayer 3 is connected to the pressure manometer 134 of the air outlet 132 of the compressor 1 by a tube 33. Thus, the mist sprayer 3 is connected to the air outlet 132 of the compressor 1. The water contained in the water chamber 31 of the mist sprayer 3 is misted by the air thrust force produced in the air outlet 132 of the compressor 1, and the mist produced in the water chamber 31 of the mist sprayer 3 is ejected outward from the spraying nozzle 32 of the mist sprayer 3.

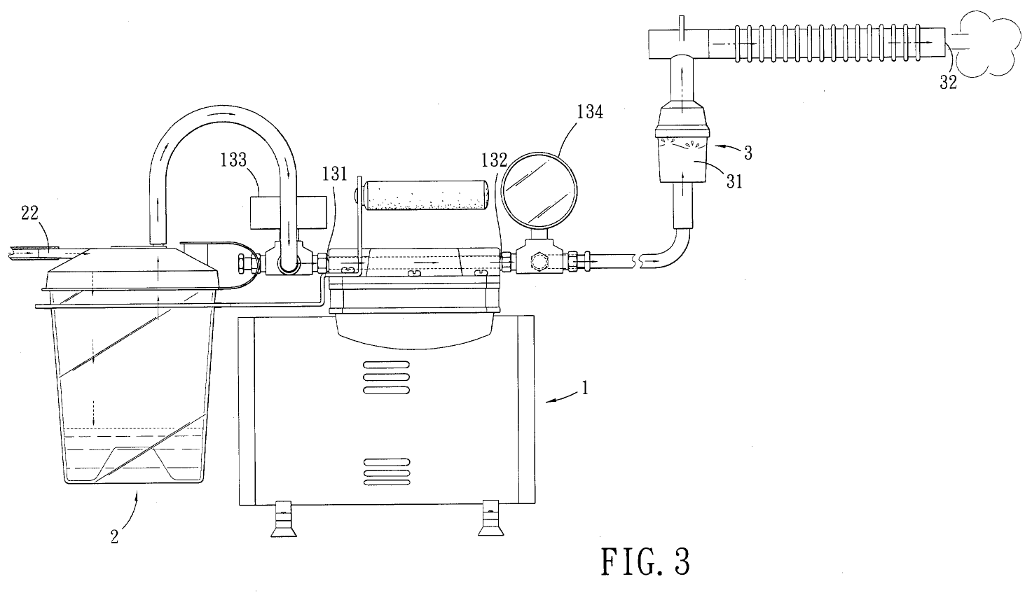

In operation, referring to FIG. 3 with reference to FIGS. 1 and 2, when the compressor 1 is started, the air extraction port 131 of the compressor 1 produces a suction force, so that the air contained in the phlegm storage container 2 is drawn outward rapidly, thereby producing a vacuum pressure. Thus, the phlegm suction port 22 of the phlegm storage container 2 produces a suction force which is directed toward the direction as indicated by the arrows, so as to suck and draw the phlegm or dirt contained and choked in the patient's throat, mouth, nose etc. through the phlegm suction port 22 of the phlegm storage container 2, thereby preventing the phlegm or dirt from choking the patient's respiratory duct. In addition, when the compressor 1 is started, the air outlet 132 of the compressor 1 can produce an air thrust force which is directed toward the direction as indicated by the arrows, so that the water contained in the water chamber 31 of the mist sprayer 3 is misted by the air thrust force produced in the air outlet 132 of the compressor 1, and the mist produced in the water chamber 31 of the mist sprayer 3 is ejected and released outward from the spraying nozzle 32 of the mist sprayer 3, so as to wet the patient's respiratory duct.

Accordingly, the composite medical equipment in accordance with the present invention can provide the phlegm extraction and mist spraying functions simultaneously, thereby reducing the cost of the equipment, and thereby facilitating the user using the composite medical equipment. In addition, the pressure manometer 133 mounted on the air extraction port 131 of the compressor 1 and the pressure manometer 134 mounted on the air outlet 132 of the compressor 1 are used to measure the pressure of the gas flowing through the air extraction port 131 and the air outlet 132 of the compressor 1, thereby preventing the phlegm storage container 2 and the mist sprayer 3 from being inoperative due to the insufficient pressure force of the air extraction port 131 and the air outlet 132 of the compressor 1.

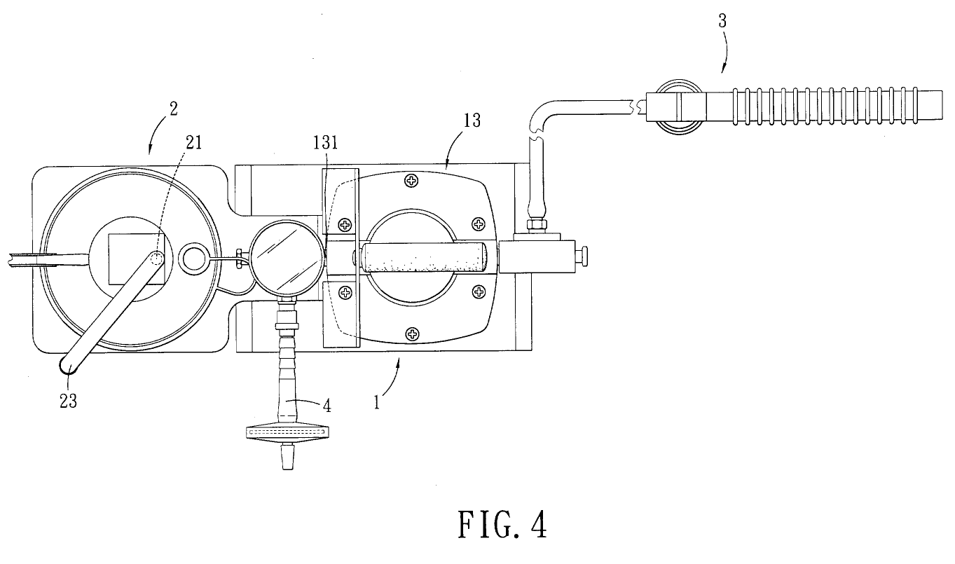

Referring to FIG. 4, the composite medical equipment having the phlegm extraction and mist spraying functions in accordance with another embodiment of the present invention is shown, wherein the tube 23 connected between the connection port 21 of the phlegm storage container 2 and the air extraction port 131 of the compressor 1 is removed, and a filter 4 is mounted on the air extraction port 131 of the compressor 1 for filtering the air. Thus, when the compressor 1 is started, the air sucked into the air extraction port 131 of the compressor 1 is filtered and cleaned by the filter 4. Thus, the mist sprayer 3 is operated individually.

While the preferred embodiment(s) of the present invention has been shown and described, it will be apparent to those skilled in the art that various modifications may be made in the embodiment(s) without departing from the spirit of the present invention. Such modifications are all within the scope of the present invention.

What is claimed is:

1. A composite medical equipment having the phlegm extraction and mist spraying functions, comprising a compressor, a phlegm storage container, and a mist sprayer, wherein:
the compressor includes a connection portion having a first end formed with an air extraction port and a second end formed with an air outlet;
the phlegm storage container is mounted on the compressor and is connected to the air extraction port of the compressor; and
the mist sprayer is mounted on the compressor and has a water chamber and a spraying nozzle, the mist sprayer is connected to the air outlet of the compressor.

2. The composite medical equipment having the phlegm extraction and mist spraying functions in accordance with claim 1, wherein the compressor can produce an air thrust force of two to three kilograms.

3. The composite medical equipment having the phlegm extraction and mist spraying functions in accordance with claim 1, wherein the compressor has a top provided with a support frame for mounting and supporting the phlegm storage container.

4. The composite medical equipment having the phlegm extraction and mist spraying functions in accordance with claim 1, wherein the compressor has a top provided with a handgrip.

5. The composite medical equipment having the phlegm extraction and mist spraying functions in accordance with claim 1, wherein the air outlet of the compressor can produce an air thrust force of two to three kilograms by the compressor.

6. The composite medical equipment having the phlegm extraction and mist spraying functions in accordance with claim 1, wherein the compressor includes a pressure manometer mounted on the air extraction port.

7. The composite medical equipment having the phlegm extraction and mist spraying functions in accordance with claim 1, wherein the compressor includes a pressure manometer mounted on the air outlet.

8. The composite medical equipment having the phlegm extraction and mist spraying functions in accordance with claim 6, wherein the phlegm storage container includes a connection port connected to the pressure manometer of the air extraction port of the compressor.

9. The composite medical equipment having the phlegm extraction and mist spraying functions in accordance with claim 1, wherein the phlegm storage container includes a connection port connected to the air extraction port of the compressor.

10. The composite medical equipment having the phlegm extraction and mist spraying functions in accordance with claim 1, wherein the phlegm storage container includes a phlegm suction port.

11. The composite medical equipment having the phlegm extraction and mist spraying functions in accordance with claim 7, wherein the mist sprayer is connected to the pressure manometer of the air outlet of the compressor by a tube.

12. The composite medical equipment having the phlegm extraction and mist spraying functions in accordance with claim 1, further comprising a filter mounted on the air extraction port of the compressor, so that the mist sprayer is operated individually.
